(19) United States
(12) Patent Application Publication
(10) Pub. No.: US 2006/0193915 A1
(43) Pub. Date: Aug. 31, 2006

(54) Compression coated tablets

(76) Inventors: Mintong Guo, Plainsboro, NJ (US); Indranil Nandi, Plainsboro, NJ (US); Ashish Anilbhai Patel, Kendall Park, NJ (US); Chuanbin Wu, Franklin Park, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/348,286

(22) Filed: Feb. 6, 2006

Related U.S. Application Data
(63) Continuation of application No. 10/263,161, filed on Oct. 2, 2002, now abandoned.

(51) Int. Cl. A61K 31/404; A61K 9/24

(52) U.S. Cl. 424/472; 514/419

(57) ABSTRACT

A pharmaceutical composition for oral administration comprising a compression coated solid dosage form of a bitter or unpleasant tasting pharmaceutically active agent. Included are compression coated oral dosage forms of 3-[2-(dimethylamino)ethyl]-N-methyl-1H-indole-5-methanesulphonamide or a pharmaceutically acceptable salt or solvate thereof as active ingredient. The compression coated solid dosage forms are of use in the treatment of conditions associated with cephalic pain, in particular migraine.

### BACKGROUND OF THE INVENTION

The present invention relates to a pharmaceutical composition containing as active ingredient 3-[2-(dimethylamino)ethyl]-N-methyl-1H-indole-5-methanesulphonamide which may be represented by the formula (I) 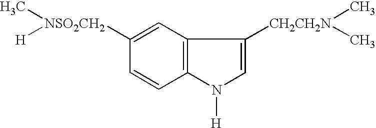
and its physiologically acceptable salts and solvates. Compounds of formula (I) are disclosed in U.S. Pat. Nos. 4,816,470 and 5,037,845. Compounds of formula (I) exhibits selective vasoconstrictor activity and is useful in the treatment of migraine. Included within the description of formula (1) is sumatriptan succinate.

Sumatriptan is useful in the treatment of conditions associated with cephalic pain, in particular migraine. However when administrated as an oral dosage form, sumatriptan's unpleasant taste and smell may exacerbate the nausea and vomiting associated with migraine.

Sumatriptan, in the form sumatriptan succinate, has very good solubility. Such good solubility makes it very difficult to mask the bitter taste of the drug because the more soluble the drug substance, the quicker it will dissolve in the mouth, and therefore it will give the patients stronger bitter taste. Therefore when intended to be administered as an oral dosage form, it is necessary to use taste masking techniques to cover the active drug substance in the final dosage form to prevent direct contact with the tongue.

The present invention discloses a bitter active pharmaceutical agent formulated as a tablet, which is compression coated by non-interacting materials. This allows the formulated dosage to be used as an oral dosage form, and provides the following advantages: a) elimination of the bitter taste and unpleasant smell of the active pharmaceutical ingredient; b) elimination of water or other solvent in the coating procedure and thereby decreasing the possible degradation of the active pharmaceutical ingredient; and c) easier and more economical manufacturing processes. Additionally the compression coatings of the present invention may include flavoring agents, which could improve the patient's compliance and acceptance with the drug regime.

### SUMMARY OF THE INVENTION

The present invention is directed to a “compression-coated solid dosage form” comprising a solid core comprising the active ingredient, which solid core is substantially covered with a compression coating. The invention is specifically directed to compression coated taste masked solid dosage forms of sumatriptan or sumatriptan succinate.

### DETAILED DESCRIPTION OF THE INVENTION

As used herein the terms “compression-coated solid dosage form” or “solid dosage form” as used herein refer to a solid core comprising the active ingredient, which solid core is substantially covered with a compression coating.

The core formulation comprises an active ingredient and any other pharmaceutically acceptable carriers or excipients. For example the core formulation may comprise a disintegrant, a filler, a suitable lubricant and the active ingredient. The core formulation may also include other components which are necessary to meet appropriate drug delivery tasks, and/or for the manufacturing purposes, such as binders, surfactant agents, wetting agents and glidants.

Suitable disintegrants include sodium starch glycollate, crospovidone, hydroxypropyl cellulose, starch, calcium carboxymethlycellulose, and croscarmellose sodium. When present, the disintegrant is present in an amount of 0 to 15% w/w, preferably 3 to 5% w/w of the core composition.

Fillers include lactose, sorbitol, mannitol, cellulose, starch, sucrose, maize starch, microcrystalline cellulose or calcium hydrogen phosphate. When present, filler comprises an amount of 5 to 99% w/w, preferably 7.5 to 50% w/w of the core composition.

Examples of lubricants include magnesium stearate, sodium stearate and stearic acid, polyethylene glycol, talc or silica. The lubricant may comprise an amount of 0 to 5% w/w, preferably 0.25 to 2% w/w of the core composition.

Examples of binders include starch, hydroxypropyl cellulose, polyvinylpyrrolidone or hydroxypropylmethylcellulose. When present, the binder is present in an amount of 0 to 20% w/w, preferably 5 to 10% w/w of the core composition.

Suitable surfactants include polysorbate, sodium lauryl sulfate and glidants include silicon dioxide and talc. When present, the surfactant is present in an amount of 0 to 5% w/w of the core composition.

The active ingredient may be any bitter or unpleasant tasting pharmaceutically active agent. It is preferred that 3-[2-(dimethylamino)ethyl]-N-methyl-1H-indole-5-methanesulphonamide represented by formula (1) above be employed in the compositions of the invention in the form of a physiologically acceptable salt. Such salts include salts of inorganic or organic acids such as hydrochloride, hydrobromide, sulphate, nitrate, phosphate, formate, mesylate, citrate, benzoate, fumarate, maleate, tartrate and succinate salts. Most preferably 3-[2-(dimethylamino)ethyl]-N-methyl-1H-indole-5-methanesulphonamide will be employed in the compositions of the invention in the form of its succinate (1:1) salt.

The amount of 3-[2-(dimethylamino)ethyl]-N-methyl-1H-indole-5-methanesulphonamide, preferably in the form of a physiologically acceptable salt, employed in the compositions of the invention will preferably be in the range of about 25 mg to about 200 mg, most preferably about 35, 70, or 140 mg of sumatriptan succinate which is equivalent to 25 mg, 50 mg or 100 mg of sumatriptan expressed as the weight of free base.

The non-interacting compression coating comprises a filler. The coating may also optionally comprise lubricant and/or disintegrant. The compression coating may also comprise a flavoring agent.

Examples of fillers include microcrystalline cellulose, lactose, starch, mannitol, dibasic calcium phosphate dihydrate, sorbitol, and calcium carbonate. Fillers will comprise about 25 to 100% w/w of the coating formulation.

Lubricants include magnesium stearate, stearic acid, sodium stearate and hydrogenated vegetable oil. Lubricants will comprise about 0 to 25% w/w, preferably 0.1 to 2% w/w of the coating formulation.

Examples of suitable disintegrants include starch, sodium starch glycolate, crospovidone. Disintegrants will comprise about 0 to 30% w/w of the coating formulation.

Examples of flavoring agents include fruit punch, raspberry, orange and grape, and are present in amounts of 0 to 15% w/w, preferably 1 to 5% w/w of the coating formulation.

The total compression coating comprises an amount of from 20 to 95% w/w, preferably from 50 to 70% w/w, based on the total weight of the solid dosage form. The compression coating of the present invention may have a thickness of 0.1 to 5 mm.

The compression coating may optionally comprise additional pharmaceutically acceptable colorants or opacifiers including azo dyes, water soluble dyes, aluminium lakes of water soluble dyes and inorganic pigments such as titanium dioxide and iron oxide. Suitable colorants or opacifiers may comprise from 0.01% to 5% w/w, preferably from 0.1 to 0.5% w/w, based on the weight of the coating formulation.

A preferred embodiment of the present invention is a compression coated tablet comprising a tablet core containing an effective amount of 3-[2-(dimethylamino)ethyl]-N-methyl-1H-indole-5-methanesulphonamide or a pharmaceutically acceptable salt or solvate thereof as active ingredient and a compression coat on the tablet core.

For the preparation of compositions according to the invention, 3-[2-(dimethylamino)ethyl]-N-methyl-1H-indole-5-methanesulphonamide or a physiologically acceptable salt or solvate thereof may be blended with suitable excipients and, if desired, granulated. Preferably 3-[2-(dimethylamino)ethyl]-N-methyl-1H-indole-5-methanesulphonamide will be granulated with a filler before admixture with the other excipients. Most preferably the filler employed will be lactose. Cores or tablets in uncoated form may be prepared, for example, by compression of the powder blend or granulate, using a lubricant as a tableting aid.

The coating formulation may be applied to the core by standard compression techniques which involve compressing coating material about the core. The result is a layer of coating material around a central core. Typically the coating is applied using a compression coating machine. Since the coating is compressed around the core in a manner similar to that used to make a tablet, tableting machinery may also be used to apply the compression coating to the core.

Depending on the principles of the compression-coating machinery, the core can be made in one tablet forming machine and then transferred to another tableting machine for application of the coat. The core may also be transferred to a compression-coating machine for the compression coating process. Examples of such machinery include Colton Model 232 and Stokes Model 538. Alternatively the core can be made on one side of the machine, and instantaneously transferred to the other side of the machine for the application of compression coating. An example of this type of machinery is the Manesty DryCota tableting machine from Casburt Limited.

After the compression coating, the final solid dosage form includes the core tablet and the coating layer which covers or conceals the core tablet.

The compositions of the invention may comprise, for example, granules, tablets or capsules. Preferably the compositions of the invention will comprise tablets, most preferably compressed tablets.

It has been found that the unpleasant taste associated with oral administration of the compound of formula (I) may substantially eliminated by the formulations of the present invention. There are numerous advantages of the present invention compared to the prior art film coated tablets. The compression coating makes the formulations easier to handle and less expensive to produce compared to film coated tablets. The materials used to prepare a compression coat are less expensive than those used for film coating. The processing and machinery used for compression coating are faster and can run at higher capacity than that used for film coating. Further, compression coating allows for the addition of flavor to the coating, and because the thickness of the compression coat can be adjusted, the amount of flavor added can also be adjusted. Such flavoring improves taste and may enhance patient compliance with the prescribed drug regimen. Further since there is no water utilized in the compression coating process, the potential degradation of the active ingredient is reduced. Surprisingly these advantages are attained without any significant loss in the bioavailability of the active compound which remains similar to film coated tablets, aqueous solutions or dispersible tablet formulations used for oral administration to treat migraines.

A further aspect of the invention provides a method of treating a mammal, including man, suffering from or susceptible to conditions associated with cephalic pain such as cluster headache, chronic paroxysmal hemicrania, headache associated with vascular disorders, headache associated with substances or their withdrawal (for example drug withdrawal), tension headache and in particular migraine which comprises oral administration of a pharmaceutical composition comprising a compression-coated solid dosage form of 3-[2-dimethylamino)ethyl]-N-methyl-1H-indole-5-methanesulphonamide or a pharmaceutically acceptable salt or solvate thereof as active ingredient. The method may treat established symptoms or may be used prophylactically to prevent symptoms.

The precise therapeutic dose of the active ingredient will depend on the age and condition of the patient and the nature of the condition to be treated and will be determined by the attending physician or health care provider.

However, in general effective doses for the treatment of conditions associated with cephalic pain, for example acute treatment of migraine, will lie in the range of 10 to 500 mg, preferably 20 to 300 mg, most preferably 25 to 200 mg, for example 25 mg, 50 mg or 100 mg of the active ingredient per unit dose which could be administered in single or divided doses, for example, 1 to 4 times per day. Preferred final dosage forms will comprise about 35, 70, or 140 mg of sumatriptan succinate which is equivalent to 25 mg, 50 mg or 100 mg of sumatriptan expressed as the weight of free base.

The invention will now be illustrated by the following Examples, which are merely illustrative and not intended to limit the scope of the present invention.

### EXAMPLE 1

### Compression Coated Tablet

Tablet cores

| (1) Sumatriptan succinate* | 70 | mg |
|---|---|---|
| (2) Lactose | 58 | mg |
| (3) Microcrystalline cellulose | 16 | mg |
| (4) Croscarmellose Sodium | 4.5 | mg |
| (5) Magnesium stearate | 1.5 | mg |
| (6) Purified water | Qs | |
| Total | 150 | mg |

*Equivalent to 50 mg free base.

Compression coating layer

| | | |
|---|---|---|
| (1) Lactose | 127.5 | mg |
| (2) Microcrystalline cellulose | 120.0 | mg |
| (3) Magnesium Stearate | 2.5 | mg |
| Total | 250 | mg |

(per tablet)

Sumatriptan succinate, 70 mg, is blended with 58 mg lactose regular and 16 mg microcrystalline. The mixture is granulated and a suitable amount of water is added during the granulation process. The granulation, after drying and milling, is mixed with croscarmellose sodium and magnesium stearate. The resulting granulate is compressed into a tablet core.

The compression coating formulation is prepared by blending 127.5 mg lactose with 120 mg microcrystalline cellulose, and then mixing with 2.5 mg magnesium stearate. The obtained powder is then compression coated around the tablet core using a Manesty DryCota tableting machine from Casburt Limited.

### EXAMPLE 2

### Compression Coated Tablet with Flavoring Agent

Tablet cores

| (1) Sumatriptan succinate* | 70 | mg |
|---|---|---|
| (2) Sorbitol | 66.5 | mg |
| (3) Povidone | 4.5 | mg |
| (4) Croscarmellose Sodium | 7.5 | mg |
| (5) Magnesium stearate | 1.5 | mg |
| (6) Purified water | Qs | |
| Total | 150 | mg |

*Equivalent to 50 mg free base.

Compression coating layer

| | | |
|---|---|---|
| (1) Sorbitol | 237.5 | mg |
| (2) Flavor agent | 10.0 | mg |
| (3) Magnesium Stearate | 2.5 | mg |
| Total | 250 | mg |

(per tablet)

Sumatriptan succinate, 70 mg, is blended with 66.5 mg sorbitol and 4.5 mg povidone. The mixture is granulated and a suitable amount of water is added during the granulation process. The granulation, after drying and milling, is mixed with croscarmellose sodium and magnesium stearate. The resulting granulate is compressed into a tablet core.

The compression coating formulation is prepared by blending 237.5 mg sorbitol with 10.0 mg of a flavoring agent, and then mixing with 2.5 mg magnesium stearate. The obtained powder is then compression coated around the tablet core using a Manesty DryCota tableting machine from Casburt Limited.

1. (canceled)

2. A solid dosage form comprising a core of 3-[2-(dimethylamino)ethyl]-N-methyl-1H-indole-5-methanesulphonamide succinate (1:1) salt and a pharmaceutically acceptable excipient, wherein said core is substantially covered with a compression coating which conceals said core.

3. A pharmaceutical composition as claimed in claim 2 wherein the compression coating comprises a filler.

4. A pharmaceutical composition as claimed in claim 3 wherein the compression coating comprises a disintegrant and a lubricant.

5. A pharmaceutical composition as claimed in claim 2 wherein the compression coating comprises 20 to 95% w/w based on the weight of the solid dosage form.

6. A pharmaceutical composition as claimed in claim 2 wherein the compression coating comprises 50 to 70% w/w based on the weight of the solid dosage form.

7-10. (canceled)

11. A method of treating a mammal being susceptible to or suffering from migraine which comprises administering a solid dosage form comprising a core of 3-[2-(dimethylamino)ethyl]-N-methyl-1H-indole-5-methanesulphonamide succinate (1:1) salt and a pharmaceutically acceptable excipient, wherein said core is substantially covered with a compression coating which conceals said core.
